(19) United States
(12) Patent Application Publication
(10) Pub. No.: US 2026/0224405 A1
(43) Pub. Date: Aug. 6, 2026

(54) SPORTS GOGGLES

(71) Applicant: Racing Force International W.L.L., Sakhir (BH)

(72) Inventors: Alexandros MIOTTO HARISTOS, Amsterdam (NL); Oscar TOFANELLI, Viareggio (IT)

(21) Appl. No.: 19/152,251

(22) PCT Filed: Jan. 31, 2024
(86) PCT No.: PCT/IB2024/050883

§ 371 Date: Jul. 30, 2025

(30) Foreign Application Priority Data
Jan. 31, 2023 (IT) 102023000001524

(51) Int. Cl. A61F 9/02; H04N 23/57

(52) U.S. Cl. CPC A61F 9/029; A61F 9/026; A61F 9/027; H04N 23/57

(57) ABSTRACT

Sports goggles which include a frame with an anatomical and ergonomic shape, one or more lenses externally attached to said frame, a possible internal padding attached to said frame, and a possible adjustment strap attached at the ends to said frame in order to adjust the adhesion of the sports goggles to the face. The sports goggles include image acquisition means and audio acquisition means associated with said frame and/or with said padding for video recording of sports activity.

### FIELD OF THE INVENTION

The present invention concerns sports goggles that can be used, for example, in winter sports such as skiing or snowboarding, motocross or BMX, soft-air, and/or other types of contact sports such as football, basketball, or rugby, where it is necessary or preferable to protect the eyes from weathering, such as rain, snow, or wind, from external agents, such as mud or other splashes, from accidental contact occurring during sports practice, or to improve visibility.

### BACKGROUND OF THE INVENTION

In the practice of sports such as skiing, snowboarding, motocross, BMX, soft-air, and others, in addition to the use of a protective helmet, sports goggles, or sports masks, are also used to protect the eyes from the weather, external agents, or to improve the visibility of the sportsman.

In addition, nowadays, for the aforementioned sports, there is an increasing desire for first-person video recording of sports activity, whether in a competitive context to make the experience of the end viewer more immersive and engaging, or in an amateur context, for example, to publish the own video recording in streaming.

Usually, video recording is made with devices preferably attached to helmets or other body parts.

Although devices of this type are popular in the marketplace, they have limitations due to their exposure, so they can become detached, bend, or break, for example, due to an accidental drop.

In addition, the point of view, or taking, of the video recording is shifted, normally upward, from the real visual of those who practice the sports activity, basically coinciding with the position of sports goggles; therefore, the video recording may be unsatisfactory and less realistic for the final viewer.

There is therefore the need to perfect sports goggles that can overcome at least one of the disadvantages of the state of the art, particularly so as to provide a shot as close as possible to the real visual of those who practice the sports activity.

In particular, one purpose of the present invention is to make sports goggles that can acquire images as closely as possible corresponding to the real visual of the user, or sportsman.

Another purpose of the present invention is to make sports goggles that ergonomically and safely integrate such means of video recording sports activity.

A further purpose of the present invention is to make sports goggles whose weight and size are not altered from the sports goggles already on the marketplace.

The Applicant has devised, tested and embodied the present invention to overcome the shortcomings of the state of the art and to obtain these and other purposes and advantages.

### SUMMARY OF THE INVENTION

The present invention is set forth and characterized in the independent claims. The dependent claims describe other characteristics of the present invention or variants to the main inventive idea.

In accordance with the above purposes and to resolve the technical problem disclosed above in a new and original way, also achieving considerable advantages compared to the state of the prior art, sports goggles are made according to the present invention, which can be used in sports such as skiing or snowboarding, motocross or BMX, soft-air, or other contact sports such as football, basketball, or rugby, comprising a frame with an anatomical and ergonomic shape, one or more lenses externally attached to said frame, a possible internal padding attached to said frame, and a possible adjustment strap attached at the ends to said frame in order to adjust the adhesion of the sports goggles to the face.

In accordance with one aspect of the present invention, the sports goggles also comprise image acquisition means and audio acquisition means associated with said frame and/or with said padding, and electronic means for processing data acquired by said image and audio acquisition means, connected to electric power supply means. In particular, at least said image acquisition means are positioned in such a way as to be disposed in proximity to the eye zone, or in the visual field, of said user, in order to acquire images that have a visual perspective comparable to the visual perspective of the user.

A visual field can be understood, here and in the following description and claims, to mean a band that includes the eyes and part of the nose and mouth of the user of the sports goggles, so that the acquired images, particularly when taken by multiple cameras whose signal can then be combined, represent in a highly realistic way what the sportsman sees during his activity.

In accordance with another aspect of the present invention, said image acquisition means comprise one or more image acquisition sensors and said audio acquisition means comprise at least one microphone. Said electronic processing means comprise at least one image processing device and a program processor operatively connected to each other.

Specifically, image acquisition sensors are arranged, associated with said frame and/or padding, behind said one or more lenses.

In accordance with another aspect of the present invention, said electronic processing means also comprise a serialization device and a deserialization device connected to each other by means of a connection cable and to one or more other electronic processing means.

In accordance with another aspect of the present invention, one or more of said electronic processing means can be disposed in an external container that is separate from said frame and/or from said padding. In particular, said power supply means can also be positioned in said container.

In accordance with another aspect of the present invention, said container is removably attached on said adjustment strap, in correspondence with a rear zone thereof, opposite to said frame.

This arrangement makes it possible to ensure safe conditions for the sportsman, both mechanically and thermally.

In accordance with another aspect of the present invention, said electronic processing means comprise, connected in sequence, said serialization device, said deserialization device, said image processing device and said program processor, wherein said serialization device is associated with the frame and/or with said padding and is positioned in a suitable housing seating, and wherein said deserialization device, said image processing device and said program processor are disposed in said container.

In accordance with another aspect of the present invention, said electronic processing means comprise, connected in sequence, said image processing device, said serialization device, said deserialization device and said program processor, wherein said image processing device and said serialization device are associated with said frame and/or with said padding and are positioned in a suitable housing seating, and wherein said deserialization device and said program processor are disposed in said container.

In accordance with another aspect of the present invention, at least said image processing device and said program processor are associated with said frame and/or with said padding and are positioned in a suitable housing seating. In addition, said power supply means are also positioned in said housing seating.

In accordance with another aspect of the present invention, at least one image acquisition sensor is positioned in correspondence with an upper central zone of said frame.

In accordance with another aspect of the present invention, at least one image acquisition sensor is positioned in correspondence with a lower central zone of said frame.

In accordance with another aspect of the present invention, which can be combined with one or more of the preceding aspects, one or more image acquisition sensors are positioned in correspondence with one or more external lateral zones of said frame, to the side of the user's eyes. In addition, in order to achieve a stereoscopic vision in video recording, at least two image acquisition sensors are positioned specularly in said external lateral zones.

In accordance with a further aspect of the present invention, one or more image acquisition sensors are positioned in correspondence with one or more lower lateral zones of said frame, under the user's eyes. In addition, in order to achieve a stereoscopic vision in video recording, at least two image acquisition sensors are positioned specularly in said lower lateral zones.

### DESCRIPTION OF THE DRAWINGS

These and other aspects, characteristics and advantages of the present invention will become apparent from the following description of some embodiments, given as a non-restrictive example with reference to the attached drawings wherein:

FIG. 1 is a front axonometric view of sports goggles according to the present invention;

FIG. 2 is a rear axonometric view of sports goggles according to the present invention;

FIGS. 3 to 6 are frontal views of sports goggles according to the present invention, in accordance with a first, second, third, and fourth variant;

FIGS. 7 to 9 are constructional schematizations of the sports googles of FIG. 1, according to three different embodiments.

We must clarify that the phraseology and terminology used in the present description, as well as the figures in the attached drawings also in relation as to how described, have the sole function of better illustrating and explaining the present invention, their purpose being to provide a non-limiting example of the invention itself, since the scope of protection is defined by the claims.

To facilitate comprehension, the same reference numbers have been used, where possible, to identify identical common elements in the drawings. It is understood that elements and characteristics of one embodiment can be conveniently combined or incorporated into other embodiments without further clarifications.

### DESCRIPTION OF SOME EMBODIMENTS OF THE PRESENT INVENTION

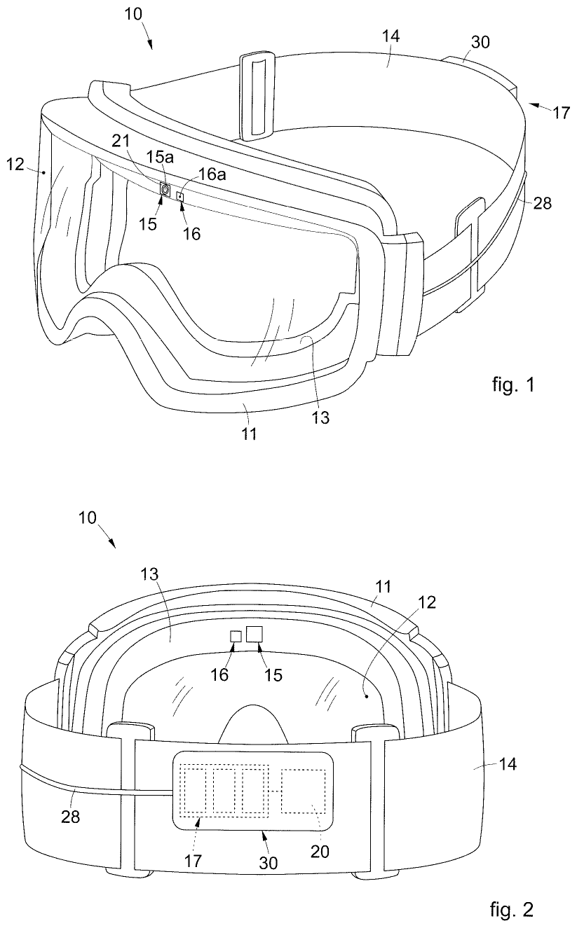

With reference to FIG. 1, sports goggles 10 according to the present invention, can be used in winter sports such as skiing or snowboarding, motocross or BMX, or soft-air and/or other types of contact sports such as football, basketball, or rugby, where it is necessary, or preferable, to use sports goggles, or sports masks, including in combination with a protective helmet.

Specifically, the sports goggles 10 are conformed to be placed on a user's face to protect the eyes and at least part of the face from weathering, such as rain, snow, or wind, from external agents, from accidental contact that may occur during sports practice, or to improve the visibility of the user practice the sport.

The sports goggles 10 essentially comprise a frame 11 with an anatomical and ergonomic shape, one or more lenses 12 externally attached to the frame 11, designed to protect the eyes and part of the user's face and to allow for proper and/or improved visibility, a possible internal padding 13 attached to the frame 11 and preferably made of foam to make the wearability of sports goggles 10 comfortable, and a possible adjustment strap 14 attached at the ends to the frame 11 to adjust the stability and adherence of sports goggles 10 to the face.

According to possible embodiments, the sports goggles 10 can have different shapes or sizes depending on the manufacturer, or the type of sport for which it was made.

In the embodiment shown and described herein by way of example, the sports goggles 10 are conformed as a sports mask usable in winter sports. It should be understood, however, that the invention as described and claimed herein is applicable to any type of sports goggles, or sports masks, having characteristics similar or assimilable to goggles, or masks, for winter sports.

The sports goggles 10 comprise image acquisition means 15 and audio acquisition means 16 associated with the frame 11 and/or with the padding 13 for video recording of the activity.

The image and audio acquisition means 15, 16 are connected to electronic processing means 17 for processing data acquired (FIGS. 2 and 7 to 9). This connection can be achieved by a physical link, such as by one or more cables, as will be described in detail below.

In addition, the electronic processing means 17 are connected to electric power supply means 20, such as one or more rechargeable batteries, which power both the electronic processing means 17 and the image and audio acquisition means 15, 16.

By way of example, not limited to, rechargeable batteries may be NI-MH, NI-CD, Lithium, Li-PO, or similar types.

The power supply means 20 may also be placed directly on an equipment, or vehicle, if present, or the same power supply as the equipment, or vehicle, may be exploited.

According to possible embodiments, the image acquisition means 15 can comprise one or more image acquisition sensors 15a.

The image acquisition sensors 15a can be of a known type, e.g. CCD or CMOS sensors, or one that will be realized in the future. By way of example, the image acquisition sensors 15a can have an image resolution of up to 48 Mp, may have HDR technology and high framerates (FPS), e.g. greater than or equal to 240 Hz, and/or other suitable features.

The image acquisition means 15 (FIGS. 1 to 6) are positioned in such a way as to be disposed in proximity to the eye zone, or in the visual field, in order to acquire images that have a visual perspective comparable to the visual perspective of the user of the sports goggles 10.

Specifically, the image acquisition sensors 15a are disposed, associated with the frame 11 and/or padding 13, behind the lenses 12.

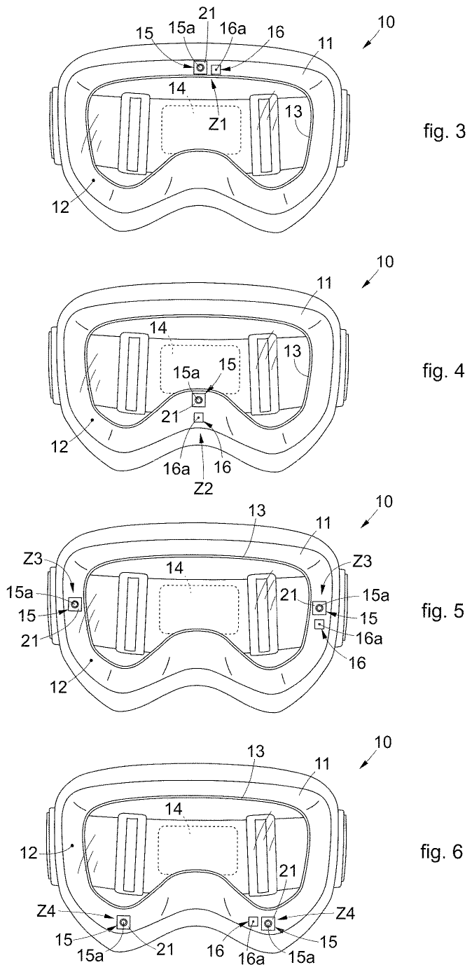

In accordance with a first implementation variant, shown in FIG. 3, at least one image acquisition sensor 15a is positioned in correspondence with an upper central zone Z1 of the frame 11.

In accordance with a second implementation variant, shown in FIG. 4, at least one image acquisition sensor 15a is positioned in correspondence with a lower central zone Z2 of the frame 11, which is superimposed on the point where the sports goggles 10 rests on the user's nose.

In accordance with a third implementation variant, shown in FIG. 5, one or more image acquisition sensors 15a are positioned in correspondence with one or more external lateral zones Z3 of the frame 11, to the side of the user's eyes. In order to achieve a stereoscopic vision, at least two image acquisition sensors 15a are positioned specularly in the external lateral zones Z3 of the frame 11.

In accordance with a fourth implementation variant, illustrated in FIG. 6, one or more image acquisition sensors 15a are positioned in correspondence with one or more lower lateral zones Z4 of the frame 11, under the user's eyes. In order to achieve a stereoscopic vision, at least two image acquisition sensors 15a are positioned specularly in the lower lateral zones Z4 of the frame 11.

It should be understood that all the implementation variants described above represent only examples of possible arrangements of image acquisition sensors, possibly combinable with each other, it being understood that other solutions not shown here in detail are still possible.

The image acquisition means 15 are attached to, or inserted into, a support 21 that is in turn attached to the frame 11 and/or padding 13. The support 21 can be made of rigid material, or of soft material, to better suit various approval and safety requirements.

According to possible embodiments, which are not represented in the drawings, more image acquisition sensors 15a can be placed in the same zone Z1, Z2, Z3, Z4, or at least one image acquisition sensor 15a in two different zones Z1, Z2, Z3, Z4; in these cases, image acquisition sensors 15a can be oriented either outward to frame the surrounding environment and inward to frame the user's eyes zone.

Specifically, the image acquisition sensors 15a directed toward the user's eyes may be suitable for either video recording, or streaming, or for use in detecting, or tracking, eyes.

In addition, according to possible forms of implementation, at least one IMU sensor, known to experts in the field, may also be installed in close proximity to a corresponding image acquisition sensor 15a, so that the data can be used to stabilize images.

According to possible embodiments, the audio acquisition means 16 comprise at least one microphone 16a.

The microphone 16a may be a known type, such as a MEMS or ECM microphone, or one that will be made in the future. By way of example, the microphone 16a may be analog or digital, may be unidirectional or omnidirectional, may have noise cancellation technology, may have a high sensitivity level, e.g. up to 145 dB SPL1 , 1 kHZ, and/or other suitable features.

By way of example, the audio acquisition means 16 are arranged in the same zone as image acquisition means 15. Possibly the audio acquisition means 16 are associated with the same support 21.

The image and audio acquisition means 15, 16 may be associated with electronic means for their operation, which are not represented in the drawings.

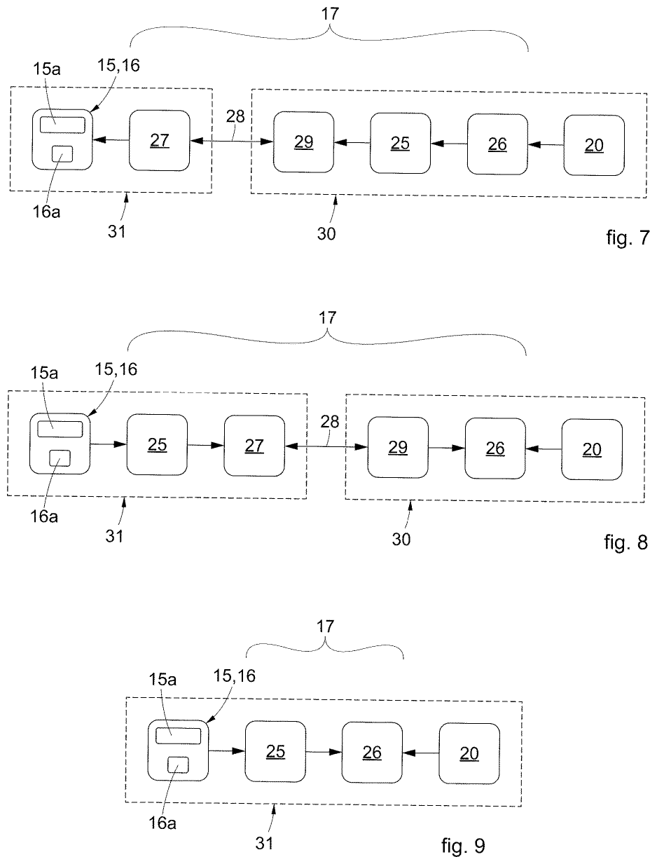

According to possible embodiments, the electronic processing means 17 (FIGS. 7 to 9) comprise at least one image processing device 25 and an and a program processor 26 connected to each other. In addition, the electronic processing means 17 may also comprise a serialization device 27 (FIGS. 7 and 8) and a deserialization device 29 connected to each other and to one or more of the other electronic processing means 17, as will be described in detail below.

In accordance with possible embodiments, one or more of the electronic processing means 17 may be associated with, or attached to, the frame 11 and/or the padding 13 within a suitable housing seating 31 (FIGS. 7, 8 and 9). Such a housing seating 31 can be made in a part of the frame 11 and/or by suitably shaping the padding 13.

According to other embodiments, one or more of the electronic processing means 17 may be arranged in an outer bag, or external container, 30 that is detached, or separated, from the frame 11 and/or padding 13. In addition, the power supply means 20 are also positioned within the container 30 (FIGS. 2 and 7 to 9).

Preferably, the container 30 is removably fixed on the adjustment strap 14 at a rear zone opposite the frame 11.

In other embodiments, the container 30 can be fixed along the entire adjustment strap 14.

In other embodiments, not depicted in the drawings, the container 30 can be attached to the helmet of the user of the sports goggles 10, or to other parts, or suitable zones.

This has the advantage of having both one or more electronic processing means 17 and the power supply means 20 detached, or separated, from the frame 11, i.e., the eye zones, to leave space for the image and audio acquisition means 15, 16.

The attachment of container 30 to the adjustment strap 14 can be achieved by means of suitable quick-connect and quick-release fasteners, not shown in the drawings.

As an example, the container can be made of soft plastic, or rubber.

The serialization device 27 is set up to receive the image and audio data acquired from the image and audio acquisition means 15, 16, to serialize them and channel them to the deserialization device 29 by means of a connection cable 28. The data received by the serialization device 27 can be raw data, that is, data acquired from the image and audio acquisition means 15, 16, or it can be already processed by the image processing device 25.

Specifically, the deserialization device 29 is set up to receive data from the serialization device 27 and to send it, once processed, to the image processing device 25, or to the program processor 26.

The connection cable 28 allows to transmit, or carry:
the voltage for the operation of the image and audio acquisition means 15, 16 and at least the serialization device 27;
the raw data, or RAW, from the serialization device 27 to the deserialization device 29; and
the control data from the deserialization device 29 to the serialization device 27.

According to possible embodiments, the connection cable 28 can be coaxial type, or a twisted pair. In addition, can be present more than one connection cable 28.

The image processing device 25 is configured to process at least the data acquired from the image acquisition means 15. For example, the image processing device 25 can adjust exposure, white balance, distortion correction, image stabilization, output video enhancement or reduction, and related compression. The data processed by image processing device 25 can be raw, or serialized and deserialized.

The program processor 26 is configured to receive image data and audio data to realize merge and combine them into a single video file, or alternatively, to realize two separate files. The received image data and/or audio data can already be processed.

In addition, the program processor 26 can receive remote commands to control the image acquisition means 15 via a wireless connection, such as via Wi-Fi, cellular network, Bluetooth, or via a wired connection, such as via USB cable, HDMI cable, or Ethernet cable.

The program processor 26 can transmit a real-time audio/video stream with low latency by exploiting wireless links, e.g., via Wi-Fi, Bluetooth, Proprietary Protocols, or by cable, e.g., via USB cable, HDMI, Ethernet, or other.

The audio/video streaming can be adapted to make itself compatible with current broadcast standards.

In addition, the program processor 26 can send data, or files, in either uncompressed RAW format or compressed format.

In addition, the program processor 26 may be able to store compressed files of type h264, h265, or future codecs, either internally, i.e., in an internal memory, or on removable means, such as Micro-SD, SD, or other.

According to possible embodiments, the program processor 26 can also be equipped with an IMU sensor system for sensing data of acceleration, magnetic field, angular velocity and others; the latter data, as with audio/video data can be transmitted in real time and/or stored on a physical medium.

In accordance with a first embodiment of the present invention (FIG. 7), the electronic processing means 17 comprise, connected in sequence, the serialization device 27, the deserialization device 29, the image processing device 25, and the program processor 26. Specifically, in such first embodiment, the serialization device 27 is associated with the frame 11 and/or the padding 13 and is positioned in the suitable housing seating 31, instead the deserialization device 29, the image processing device 25, the program processor 26 and the power supply means 20 are disposed in the container 30.

In accordance with a second embodiment of the present invention (FIG. 8), the electronic processing means 17 comprise, connected sequentially, the image processing device 25, the serialization device 27, the deserialization device 29, and the program processor 26. Specifically, in such second embodiment, the image processing device 25 and the serialization device 27 are associated with the frame 11 and/or the padding 13 and are positioned in the suitable housing seating 31, instead the deserialization device 29, the program processor 26 and the power supply means 20 are disposed in the container 30.

In accordance with a third embodiment of the present invention (FIG. 9), the electronic processing means 17 comprise the image processing device 25 that is connected to the program processor 26. Specifically, in that third embodiment, the image processing device 25 the program processor 26 and the power supply means 20 are associated with the frame 11 and/or padding 13 and disposed in the suitable housing seating 31.

It is clear that modifications and/or additions of parts or steps may be made to the sports goggles 10 described thus far, without departing from the scope of the present invention as defined by the claims.

It is also clear that, although the present invention has been described with reference to some specific examples, a person of skill in the art will be able to achieve other equivalent forms of sports goggles, having the characteristics as set forth in the claims and hence all coming within the field of protection defined thereby.

In the following claims, the sole purpose of the references in brackets is to facilitate their reading and they must not be considered as restrictive factors with regard to the field of protection of the same claims.

1. Sports goggles (10) comprising a frame (11) with an anatomical and ergonomic shape, one or more lenses (12) externally attached to said frame (11), a possible internal padding (13) attached to said frame (11), and a possible adjustment strap (14) attached at the ends to said frame (11) in order to adjust the adhesion of the sports goggles (10) to the face, wherein they comprise image acquisition means (15) and audio acquisition means (16) associated with said frame (11) and/or with said padding (13), and electronic processing means (17) for processing data acquired by said image and audio acquisition means (15, 16), connected to electric power supply means (20), wherein at least said image acquisition means (15) are positioned in such a way as to be disposed in proximity to the eye zone, or in the visual field, of said user, in order to acquire images that have a visual perspective comparable to the visual perspective of the user.

2. Sports goggles (10) as in claim 1, wherein said image acquisition means (15) comprise one or more image acquisition sensors (15a) and said audio acquisition means (16) comprise at least one microphone (16a), wherein said electronic processing means (17) comprise at least one image processing device (25) and a program processor (26) operatively connected to each other.

3. Sports goggles (10) as in claim 2, wherein said electronic processing means (17) also comprise a serialization device (27) and a deserialization device (29) connected to each other by means of a connection cable (28) and to one or more other electronic processing means (17).

4. Sports goggles (10) as in claim 2, wherein one or more of said electronic processing means (17) can be disposed in an external container (30) that is separate from said frame (11) and/or from said padding (13), wherein said power supply means (20) can also be positioned in said container (30).

5. Sports goggles (10) as in claim 4, wherein said container (30) is removably attached on said adjustment strap (14), in correspondence with a rear zone thereof, opposite to said frame (11).

6. Sports goggles (10) as in claim 5, wherein said electronic processing means (17) comprise, connected in sequence, said serialization device (27), said deserialization device (29), said image processing device (25) and said program processor (26), wherein said serialization device (27) is associated with the frame (11) and/or with said padding (13) and is positioned in a suitable housing seating (31), and wherein said deserialization device (29), said image processing device (25) and said program processor (26) are disposed in said container (30).

7. Sports goggles (10) as in claim 5, wherein said electronic processing means (17) comprise, connected in sequence, said image processing device (25), said serialization device (27), said deserialization device (29) and said program processor (26), wherein said image processing device (25) and said serialization device (27) are associated with said frame (11) and/or with said padding (13) and are positioned in a suitable housing seating (31), and wherein said deserialization device (29) and said program processor (26) are disposed in said container (30).

8. Sports goggles (10) as in claim 2, wherein at least said image processing device (25) and said program processor (26) are associated with said frame (11) and/or with said padding (13) and are positioned in a suitable housing seating (31), wherein said power supply means (20) are also positioned in said housing seating (31).

9. Sports goggles (10) as in claim 2, wherein at least one image acquisition sensor (15a) is positioned in correspondence with an upper central zone (Z1) of said frame (11).

10. Sports goggles (10) as in claim 2, wherein at least one image acquisition sensor (15a) is positioned in correspondence with a lower central zone (Z2) of said frame (11).

11. Sports goggles (10) as in claim 2, wherein one or more image acquisition sensors (15a) are positioned in correspondence with one or more external lateral zones (Z3) of said frame (11), to the side of the user's eyes, wherein, in order to achieve a stereoscopic vision in video recording, at least two image acquisition sensors (15a) are positioned specularly in said external lateral zones (Z3).

12. Sports goggles (10) as in claim 2, wherein one or more image acquisition sensors (15a) are positioned in correspondence with one or more lower lateral zones (Z4) of said frame (11), under the user's eyes, wherein, in order to achieve a stereoscopic vision in video recording, at least two image acquisition sensors (15a) are positioned specularly in said lower lateral zones (Z4).
